SEQUENCE LISTING (24 sequences)

SEQ ID NO: 1 - DNA, 6458 bp, Homo sapiens
  CDS (2235)...(3737)
cctttggctttgaattgatcaggagacaaagataatgcatctacattttcgtcttctgttcttttattggaaataagtggcacgccccattgccttctagtcgcctccccgaagcgaagaggccgaagcgaagaggcctggtgggttgtctcaacatccttttgctgagaatcgaatacgcagccgatgaacagccaggaagggtgcaaggaaacctgaaatacaaatgttctccctgaagccctcttccctgcccaaccagaccagcaacttccaaaattctgcccgtgtttagccttgttaaaggggtgtctcactccttcagggaaagtgggaaaaggggatctgattattgaggtgtggaaggaataaataatcagtccacaaataaacaaactgtccgggattcctagagggaaggagaaatccttgaaggagatccaagtcgctccaggtctgcctgccgaataatatcatcccgaagggatcttgaaccgtttgcaatcaaccgctcacccagtcttcccacggagcgcgctccctaactcaccctacccacccaacaaaacaaaaaaaaggctgaaatatagaaaagcaacttggaggctcccagggggacgttgccaggagcaggaggcagggacagcgccctagggtcggtgttagcggccggcgccggcctgggccacgggaaacgtccacgcttggtgcccgcggtgcgcggcgctcattgcgcgcgccttcgagccaagcccccgcggaaaacaggctcgggtttctcctcgcagggcccaggaactcggctctgcctggcccgggtgggtcgctgcattgtcccggtcttctgggagtgcggggtcagcttgttagagggaatttctacctgggaaaagggagacgagtttcgaagctgaagttggtaggctgcgagtgtccacgcgggagacgaaagggggaaatagcagagtcacttcacccttttccccaaaccccacaaaactgctcgcagcgacgcggatgatctaccgaattccccgcgaattcggaggattaagttgtcagtcagcacgttgctaccttcccctctatgcactccgctgcctggctcctcggcggggagcgagggaaactcagtttgtagggtttacctctaaaacctcgataggttatccttgacgaccccgagcctggaaactccctgttgatgattaattatttgattaaataagtataacatccaggagaggccctgccattccaatccagcgcgtttgcttttgaatccattacacctgggcccccataattaggaaatctaattattcgcttcatcactcattaataagaaaaatgtcccaggatcattgctacttacaaggtctttgggagagatattttactctattaatccattctattttatatttcaaattgattttttttaacagaggaaagtggctatctttttgttttgggcatgtgggcccattcaccaaaatgtgatcataaaataaattttaataagatataactttttaaaaagttttcaagtgaagacggagtcgccgcggaggccggggcggcggggtcttagagccgacggattcctgcgctcctcgccccgattggcgccggactcctctcagctgccgggtgattggctcaaagttccgggagggggcgtggcccgaggaaagtaaaaactcgctttcagcaagaagacttttgaaacttttcccaatccctaaaagggacttggcctctttttctgggctcagcggggcagccgctcggaccccggcgcgctgaccctcggggctgccgattcgctgggggcttggagagcctcctgcgcccctcctcgcgcgggccgagggtccaccttggtccccaggccgcggcgtctccgctgggtccgcggccgcccgcctgcccgcgctgccgccgccgggtcctggagccagcgaggagcggggccggcgctgcgcttgcccggggcgcgccctccaggatgccgatccgcccggtccgctgaaagcgcgcgcccctgctcggcccgagcgacgacgaccgcgcaccctcgccccggaggctgccaggagaccggggccgcccctcccgctcccctcctctccccctctggctctctcgcgctctctcgctctcagggcccccctcgctcccccggccgcagtccgtgcgcgagggcgccggcgagccgtctcggaagcagcatgcaggcgcgctactccgtgtccgaccccaacgccctgggagtggtgccctacctgagcgagcagaattactaccgggctgcgggcagctacggcggcatggccagccccatgggcgtctattccggccacccggagcagtacagcgcggggatgggccgctcctacgcgccctaccaccaccaccagcccgcggcgcctaaggacctggtgaagccgccctacagctacatcgcgctcatcaccatggccatccagaacgcgcccgagaagaagatcaccttgaacggcatctaccagttcatcatggaccgcttccccttctaccgggagaacaagcagggctggcagaacagcatccgccacaacctctcgctcaacgagtgcttcgtcaaggtgccccgcgacgacaagaagcccggcaagggcagttactggaccctggacccggactcctacaacatgttcgagaacggcagcttcctgcggcgccggcggcgcttcaaaaagaaggacgtgtccaaggagaaggaggagcgggcccacctcaaggagccgcccccggcggcgtccaagggcgccccggccaccccccacctagcggacgcccccaaggaggccgagaagaaggtggtgatcaagagcgaggcggcgtccccggcgctgccggtcatcaccaaggtggagacgctgagccccgagagcgcgctgcagggcagcccgcgcagcgcggcctccacgcccgccggctcccccgacggttcgctgccggagcaccacgccgcggcgcccaacgggctgcctggcttcagcgtggagaacatcatgaccctgcgaacgtcgccgccgggcggagagctgagcccgggggccggacgcgcgggcctggtggtgccgccgctggcgctgccatacgccgccgcgccgcccgccgcctacggccagccgtgcgctcagggcctggaggccggggccgccgggggctaccagtgcagcatgcgagcgatgagcctgtacaccggggccgagcggccggcgcacatgtgcgtcccgcccgccctggacgaggccctctcggaccacccgagcggccccacgtcgcccctgagcgctctcaacctcgccgccggccaggagggcgcgctcgccgccacgggccaccaccaccagcaccacggccaccaccacccgcaggcgccgccgcccccgccggctccccagccccagccgacgccgcagcccggggccgccgcggcgcaggcggcctcctggtatctcaaccacagcggggacctgaaccacctccccggccacacgttcgcggcccagcagcaaactttccccaacgtgcgggagatgttcaactcccaccggctggggattgagaactcgaccctcggggagtcccaggtgagtggcaatgccagctgccagctgccctacagatccacgccgcctctctatcgccacgcagccccctactcctacgactgcacgaaatactgacgtgtcccgggacctcccctccccggcccgctccggcttcgcttcccagccccgacccaaccagacaattaaggggctgcagagacgcaaaaaagaaacaaaacatgtccaccaaccttttctcagacccgggagcagagagcgggcacgctagcccccagccgtctgtgaagagcgcaggtaactttaattcgccgccccgtttctgggatcccaggaaacccctccaaagggacgcagcccaacaaaatgagtattggtcttaaaatccccctcccctaccaggacggctgtgctgtgctcgacctgagctttcaaaagttaagttatggacccaaatcccatagcgagcccctagtgactttctgtaggggtccccataggtgtatgggggtctctatagataatatatgtgctgtgtgtaattttaaatttctccaaccgtgctgtacaaatgtgtggatttgtaatcaggctattttgttgttgttgttgttgttcagagccattaatataatatttaaagttgagttcactggataagtttttcatcttgcccaaccatttctaactgccaaattgaattcaagaaaccgatgtgggttttgtttcctgtacaattatgagatataattctttttcccattgtaggtcttttacaaaacaagaaaataatttatttttttgttggtggataaagaagtcaagtatctgatactttttatttacaaagtgtgatggttttgtatagtaggttccaccctgagtattcctaaaagaaaaaaaaaaaaaaagcttaaaaactctaacttcatctgtgtttgtcttacgtggtcttaatcgttgtacttaccttaaaataaacccatgttgttttttctgcccaaagtttggacagtgtgtttgtgttgttgcattttttacaaacgaggtgtgtttgcaaacccacctgctttgattatttttgttacacaggtgggtatatgtgtagacacataaaaacgaccagagaataggagcacacacctgctgtcttgtttagtgacagaaaaaggcttttgattaattttaaaatcccactctaggattttttcttttcgagaaaccgcccagttggagggggctgcctgaaggaccggaccatgagtttgccgtgatgcattttcttaaatgcacaaaaacatgctaattgtcaaaacaaacagtgccactccatctcagtgtccagccgtccccagtttaggaggtgaaggaagggaagaataaacatttcccgtttgctaactgcaacccagggtgagtcctgctttcccccgattttataaaatttgagcctctttgcctgctttaatagttttccagagaatttgaactgggccaatgaaggtctgaaggggacggattttctagcgtttgatatccatcccccttagcggccagatcagaggggaatttcagactttattacttctcaatgtcatgtctaaatctacaccctcatcgcagtgaaaaattttaaaacctcattacccttcaaaaataatttatgatatttttagagttctaaattcaagtttttcaatatgttaaataatagagattattttttgttttcaatgttaatatctcgtcttttacatttttaatagtaacatagtttttgtgaaatgtagctgacgaaatggctttattatctatttcaatggctgaagtccaccactcccctgctggcctctatgtgtgaatttggggaccaaagcttcatcaattcccaccccagcaggtgagctgtaccttgctaatgctgaagttctttgtgagcttaacgtttcaagaccagatgattttgctaaaggtgattttgcttgatgcagtggcgctgaacgtaacccgggtgtttttgtcgtgttgttttcaacatggcactttatctccacgctatgttgaaatagaattaggggaagcttaaagcataataattgtccccacatgtgcaacacagactctttcaatctgtggccccagaggtggcacacagttaagacttggcggctgtctcattctttttcataatgtgcgggttcccgggtgtccgggtgctagactttcagcaggccccaggccagacgggctttggttgagtgaacaggaggaggaagttaaggaggtaggggtggggagagaccctctccaagctgcagaagaaggtggcccaagctccttgcctgcgtctgccgtgatggtttcattttacttctgctcgcttcatgctatttgccccaggagaagaggagagtattccagacggtaagcgagctggctttttcccttccctagacgttttaaagaaatctttctgaaagcttgccctcatcgtaagctttgaaaccgttggtgtcctgttagtggcgagggctgagagacacgcggagaaataaaggagagcgacggtgtggctgagagcccccaggtctgctgttgaaactaagctgggcttttgcacctttaggaagcctttttaaagaagtcctgctgtgtgggggccggaagcccaagtgagtgggccttgtggaggttatcgggaggggtctttaccactccttggggaacgtgggcaacggggggattgtatctgaagctttattcaggtcttcggcggcagcagagtggagaaccaggcccttagtgtgtagcggcctggggattttgggactcatc

SEQ ID NO: 2 - protein, 501 aa, Homo sapiens
MQARYSVSDPNALGVVPYLSEQNYYRAAGSYGGMASPMGVYSGHPEQYSAGMGRSYAPYHHHQPAAPKDLVKPPYSYIALITMAIQNAPEKKITLNGIYQFIMDRFPFYRENKQGWQNSIRHNLSLNECFVKVPRDDKKPGKGSYWTLDPDSYNMFENGSFLRRRRRFKKKDVSKEKEERAHLKEPPPAASKGAPATPHLADAPKEAEKKVVIKSEAASPALPVITKVETLSPESALQGSPRSAASTPAGSPDGSLPEHHAAAPNGLPGFSVENIMTLRTSPPGGELSPGAGRAGLVVPPLALPYAAAPPAAYGQPCAQGLEAGAAGGYQCSMRAMSLYTGAERPAHMCVPPALDEALSDHPSGPTSPLSALNLAAGQEGALAATGHHHQHHGHHHPQAPPPPPAPQPQPTPQPGAAAAQAASWYLNHSGDLNHLPGHTFAAQQQTFPNVREMFNSHRLGIENSTLGESQVSGNASCQLPYRSTPPLYRHAAPYSYDCTKY

SEQ ID NO: 3 - DNA, 4158 bp, Homo sapiens
  CDS (187)...(1437)
cctttggctttgaattgatcaggagacaaagataatgcatctacattttcgtcttctgttcttttattggaaataagtggcacgccccattgccttctagtcgcctccccgaagcgaagaggccgaagcgaagaggcctggtgggttgtctcaacatccttttgctgagaatcgaatacgcagccgatgaacagccaggaagggtgcaaggaaaccttgaacggcatctaccagttcatcatggaccgcttccccttctaccgggagaacaagcagggctggcagaacagcatccgccacaacctctcgctcaacgagtgcttcgtcaaggtgccccgcgacgacaagaagcccggcaagggcagttactggaccctggacccggactcctacaacatgttcgagaacggcagcttcctgcggcgccggcggcgcttcaaaaagaaggacgtgtccaaggagaaggaggagcgggcccacctcaaggagccgcccccggcggcgtccaagggcgccccggccaccccccacctagcggacgcccccaaggaggccgagaagaaggtggtgatcaagagcgaggcggcgtccccggcgctgccggtcatcaccaaggtggagacgctgagccccgagagcgcgctgcagggcagcccgcgcagcgcggcctccacgcccgccggctcccccgacggttcgctgccggagcaccacgccgcggcgcccaacgggctgcctggcttcagcgtggagaacatcatgaccctgcgaacgtcgccgccgggcggagagctgagcccgggggccggacgcgcgggcctggtggtgccgccgctggcgctgccatacgccgccgcgccgcccgccgcctacggccagccgtgcgctcagggcctggaggccggggccgccgggggctaccagtgcagcatgcgagcgatgagcctgtacaccggggccgagcggccggcgcacatgtgcgtcccgcccgccctggacgaggccctctcggaccacccgagcggccccacgtcgcccctgagcgctctcaacctcgccgccggccaggagggcgcgctcgccgccacgggccaccaccaccagcaccacggccaccaccacccgcaggcgccgccgcccccgccggctccccagccccagccgacgccgcagcccggggccgccgcggcgcaggcggcctcctggtatctcaaccacagcggggacctgaaccacctccccggccacacgttcgcggcccagcagcaaactttccccaacgtgcgggagatgttcaactcccaccggctggggattgagaactcgaccctcggggagtcccaggtgagtggcaatgccagctgccagctgccctacagatccacgccgcctctctatcgccacgcagccccctactcctacgactgcacgaaatactgacgtgtcccgggacctcccctccccggcccgctccggcttcgcttcccagccccgacccaaccagacaattaaggggctgcagagacgcaaaaaagaaacaaaacatgtccaccaaccttttctcagacccgggagcagagagcgggcacgctagcccccagccgtctgtgaagagcgcaggtaactttaattcgccgccccgtttctgggatcccaggaaacccctccaaagggacgcagcccaacaaaatgagtattggtcttaaaatccccctcccctaccaggacggctgtgctgtgctcgacctgagctttcaaaagttaagttatggacccaaatcccatagcgagcccctagtgactttctgtaggggtccccataggtgtatgggggtctctatagataatatatgtgctgtgtgtaattttaaatttctccaaccgtgctgtacaaatgtgtggatttgtaatcaggctattttgttgttgttgttgttgttcagagccattaatataatatttaaagttgagttcactggataagtttttcatcttgcccaaccatttctaactgccaaattgaattcaagaaaccgatgtgggttttgtttcctgtacaattatgagatataattctttttcccattgtaggtcttttacaaaacaagaaaataatttatttttttgttggtggataaagaagtcaagtatctgatactttttatttacaaagtgtgatggttttgtatagtaggttccaccctgagtattcctaaaagaaaaaaaaaaaaaaagcttaaaaactctaacttcatctgtgtttgtcttacgtggtcttaatcgttgtacttaccttaaaataaacccatgttgttttttctgcccaaagtttggacagtgtgtttgtgttgttgcattttttacaaacgaggtgtgtttgcaaacccacctgctttgattatttttgttacacaggtgggtatatgtgtagacacataaaaacgaccagagaataggagcacacacctgctgtcttgtttagtgacagaaaaaggcttttgattaattttaaaatcccactctaggattttttcttttcgagaaaccgcccagttggagggggctgcctgaaggaccggaccatgagtttgccgtgatgcattttcttaaatgcacaaaaacatgctaattgtcaaaacaaacagtgccactccatctcagtgtccagccgtccccagtttaggaggtgaaggaagggaagaataaacatttcccgtttgctaactgcaacccagggtgagtcctgctttcccccgattttataaaatttgagcctctttgcctgctttaatagttttccagagaatttgaactgggccaatgaaggtctgaaggggacggattttctagcgtttgatatccatcccccttagcggccagatcagaggggaatttcagactttattacttctcaatgtcatgtctaaatctacaccctcatcgcagtgaaaaattttaaaacctcattacccttcaaaaataatttatgatatttttagagttctaaattcaagtttttcaatatgttaaataatagagattattttttgttttcaatgttaatatctcgtcttttacatttttaatagtaacatagtttttgtgaaatgtagctgacgaaatggctttattatctatttcaatggctgaagtccaccactcccctgctggcctctatgtgtgaatttggggaccaaagcttcatcaattcccaccccagcaggtgagctgtaccttgctaatgctgaagttctttgtgagcttaacgtttcaagaccagatgattttgctaaaggtgattttgcttgatgcagtggcgctgaacgtaacccgggtgtttttgtcgtgttgttttcaacatggcactttatctccacgctatgttgaaatagaattaggggaagcttaaagcataataattgtccccacatgtgcaacacagactctttcaatctgtggccccagaggtggcacacagttaagacttggcggctgtctcattctttttcataatgtgcgggttcccgggtgtccgggtgctagactttcagcaggccccaggccagacgggctttggttgagtgaacaggaggaggaagttaaggaggtaggggtggggagagaccctctccaagctgcagaagaaggtggcccaagctccttgcctgcgtctgccgtgatggtttcattttacttctgctcgcttcatgctatttgccccaggagaagaggagagtattccagacggtaagcgagctggctttttcccttccctagacgttttaaagaaatctttctgaaagcttgccctcatcgtaagctttgaaaccgttggtgtcctgttagtggcgagggctgagagacacgcggagaaataaaggagagcgacggtgtggctgagagcccccaggtctgctgttgaaactaagctgggcttttgcacctttaggaagcctttttaaagaagtcctgctgtgtgggggccggaagcccaagtgagtgggccttgtggaggttatcgggaggggtctttaccactccttggggaacgtgggcaacggggggattgtatctgaagctttattcaggtcttcggcggcagcagagtggagaaccaggcccttagtgtgtagcggcctggggattttgggactcatc

SEQ ID NO: 4 - protein, 417 aa, Homo sapiens
MNSQEGCKETLNGIYQFIMDRFPFYRENKQGWQNSIRHNLSLNECFVKVPRDDKKPGKGSYWTLDPDSYNMFENGSFLRRRRRFKKKDVSKEKEERAHLKEPPPAASKGAPATPHLADAPKEAEKKVVIKSEAASPALPVITKVETLSPESALQGSPRSAASTPAGSPDGSLPEHHAAAPNGLPGFSVENIMTLRTSPPGGELSPGAGRAGLVVPPLALPYAAAPPAAYGQPCAQGLEAGAAGGYQCSMRAMSLYTGAERPAHMCVPPALDEALSDHPSGPTSPLSALNLAAGQEGALAATGHHHQHHGHHHPQAPPPPPAPQPQPTPQPGAAAAQAASWYLNHSGDLNHLPGHTFAAQQQTFPNVREMFNSHRLGIENSTLGESQVSGNASCQLPYRSTPPLYRHAAPYSYDCTKY

SEQ ID NO: 5 - DNA, 6021 bp, Mus musculus
  exon (1649)...(438)
ctcgagtcaaaggtagcacacataaaacctattttgctgcttcggtacgtcaagcaatgccactaaagtttcctcacccgccaaagctgaaacagtgagttctaatctctcaaagccttttgccgaaaatctaaagggggtggggggctatggtggtggcgtggggggggggtcggagaagaagaaagactgagacaaatgttttatctgtcgccttcttccctacccaaccggaccaacaacttccagaaggttctgcgaggcatagagccattccgtagggacatctcggtgcttctgaggaagcggaccgagcagggatccgatgacgactggagatgttgaaggaataaataccagtccacaaataaacaaactgtccccgggattcctagagggaaggagcacgcttgaaggtcggggaactccgagtcgctgtgcgtcaaggttggcataaaattaaaaaaaaaaaaagtccttcagttaccaggccctctaaggagcccctggtcctcagctcaccttatcaaaactcagtaaaacaaacagcctgaaatacagtcaatttacaggatcccaaagatgctgaccgcggagtgggacccacgccgggccccggcaacagctagggaagcgggtccgaggctacacagtgccgcgctcctttgcgtttccagtgacgaagccggcgatggagtgcaggcttggagctccccacgccgaacggggacaccagctcccgggggctggctgccttgtcctaacctccagacagcgctttcataggtggggagaagggagaggccgggatggatggcagggaaagctagccctcgtctatgcgggagaggagaccaggaaagcaacagttgggttcacgcgcttccctgaaccccacgaaattgtttggaggactcagatggatcacctaagtagcagcgaagacgaaggaccaatggttccttaggtgttaccttcccagtttggcattcccactaagccttccctcccagcccgaccccgtcgtgaaggggagaggaaccgaattctccaacccggcctcctttgtgggctcttcctcaacctggaagcgtcctgtgaattatccatcactgcattcaacaggccctacacgctcagtccgtttgctctgaacccattacaactaggccccgataattaagaaatctaattattcgcctcttcatccattaataataataaaaaaaaaatctccaggctctttcctacttacaaggtcttgggggcaaatctctgcccaacttcatcaattcgatgttatatttcaaactaaacttctttttattttccaaaggaacagggtttttaatttttgctctggacacgtggtctcgttaaacaaaatgtgataataaaataaaattttataagatgtaactcatttttaaaagtcctcaagttaacttgagctgggggggggggagatctggctaagagcatctgggtcttagagccgacggattcaggcgctcctcgttttgattggtgccatccttctcgcagctgccagatgattggtgcaaacttcctggagggggcgcggcctgaagaaagtaaaaactcgctttgagccagaagacttttgaaacttttcccaatccctaaaagggactttgcttctttttccgggctcggccgcgcagcctctccggaccctagctcgctgacgctgcgggctgcagttctcctggcggggcccgagagccgctgtctccttttctagcactcggaagggctggtgtcgctccacggtcgcgcgtggcgtctgtgccgccagctcagggctgccacccgccaagccgagagtgcgcggccagcggggccgcctgccgtgcacccttcaggatgccgatccgcccggtcggctgaacccgagcgccggcgtcttccgcgcgtggaccgcgaggctgccccgagtcggggctgcctgcatcgctccgtcccttcctgctctcctgctccgggcctcgctcgccgcgggccgcagtcggtgcgcgcaggcggcgaccgggcgtctgggacgcagcatgcaggcgcgttactcggtatcggaccccaacgccctgggagtggtaccctatttgagtgagcaaaactactaccgggcggccggcagctacggcggcatggccagccccatgggcgtctactccggccacccggagcagtacggcgccggcatgggccgctcctacgcgccctaccaccaccagcccgcggcgcccaaggacctggtgaagccgccctacagctatatagcgctcatcaccatggcgatccagaacgcgccagagaagaagatcactctgaacggcatctaccagttcatcatggaccgtttccccttctaccgcgagaacaagcagggctggcagaacagcatccgccacaacctgtcactcaatgagtgcttcgtgaaagtgccgcgcgacgacaagaagccgggcaagggcagctactggacgctcgacccggactcctacaacatgttcgagaatggcagcttcctgcggcggcggcggcgcttcaagaagaaggatgtgcccaaggacaaggaggagcgggcccacctcaaggagccgccctcgaccacggccaagggcgctccgacagggaccccggtagctgacgggcccaaggaggccgagaagaaagtcgtggttaagagcgaggcggcgtcccccgcgctgccggtcatcaccaaggtggagacgctgagccccgagggagcgctgcaggccagtccgcgcagcgcatcctccacgcccgcaggttccccagacggctcgctgccggagcaccacgccgcggcgcctaacgggctgcccggcttcagcgtggagaccatcatgacgctgcgcacgtcgcctccgggcggcgatctgagcccagcggccgcgcgcgccggcctggtggtgccaccgctggcactgccatacgccgcagcgccacccgccgcttacacgcagccgtgcgcgcagggcctggaggctgcgggctccgcgggctaccagtgcagtatgcgggctatgagtctgtacaccggggccgagcggcccgcgcacgtgtgcgttccgcccgcgctggacgaggctctgtcggaccacccgagcggccccggctccccgctcggcgccctcaacctcgcagcgggtcaggagggcgcgttgggggcctcgggtcaccaccaccagcatcacggccacctccacccgcaggcgccaccgcccgccccgcagccccctcccgcgccgcagcccgccacccaggccacctcctggtatctgaaccacggcggggacctgagccacctccccggccacacgtttgcaacccaacagcaaactttccccaacgtccgggagatgttcaactcgcaccggctaggactggacaactcgtccctcggggagtcccaggtgagcaatgcgagctgtcagctgccctatcgagctacgccgtccctctaccgccacgcagccccctactcttacgactgcaccaaatactgaggctgtccagtccgctccagccccaggaccgcaccggcttcgcctcctccatgggaaccttcttcgacggagccgcagaaagcgacggaaagcgcccctctctcagaaccaggagcagagagctccgtgcaactcgcaggtaacttatccgcagctcagtttgagatctcagcgagtccctctaagggggatgcagcccagcaaaacgaaatacagattttttttttaattccttcccctacccagatgctgcgcctgctcccttggggcttcatagattagcttatggaccaaacccatagggacccctaatgacttctgtggagattctccacgggcgcaagaggtctctccggataaggtgccttctgtaaacgagtgcggatttgtaaccaggctattttgttcttgcccagagcctttaatataatatttaaagttgtgtccactggataaggtttcgtcttgcccaactgttactgccaaattgaattcaagaaacgtgtgtgggtcttttctccccacgtcaccatgataaaataggtccctccccaaactgtaggtcttttacaaaacaagaaaataatttatttttttgttgttgttggataacgaaattaagtatcggatacttttaatttaggaagtgcatggctttgtacagtagatgccatctggggtattccaaaaacacaccaaaagactttaaaatttcaatctcacctgtgtttgtcttatgtgatctcagtgttgtatttaccttaaaataaacccgtgttgtttttctgcccaaagttcggacagagtctttgtgttcttgaattttaaaagggaaattgtagtaagccagttgtgattgatttttgtgatgcaggttggcctggtaacgtggatgcatatacaggttacaggacgatggagctctcgattagtaatagaaggggctcttgatttgttgaactatcccgtcctgagatatttttgttttctgctcgaggtaatctgagaaactgttctccatccacacacggacagggctgcctgagggcaacgtcctgctggcctgttaacgaaatgctttgcgggatgcagaaaactgttgccaattgtcaaaacaaaatggtgtcaccctgtctcggtgtccagctgtcctctgttagaggggagaaaccgagaaaggacaaacggcctgcagcttgctaacctcagcgtagcaggagcctgggtgagtgctcggctccctccatttccttagatgcggacttgttgcccctgttggcgttttaagagtgccagcaagaagcaaagagggttggtaggtctctggtatttaactgccggctttgggatcagattagaagtgaatttcagtctgatttatttcttaatttgggctttaaatattttactccggcgtggtggaaaaagaagccactgtgcgcctccagcatgatattttagcgctgaaatggctctggttttcagcatgctaagtaacaggagattatttttcttttgattcttgtatttcatttctttaaaaaaaaaaaaggaaatagatcgggacaaactctctaaaatgtacctggctggctggggtggggtccttaccaatctgctgcctgaaagatacagcttcagcacaggcctgcgtgttggactttaggcatatcatggattcccacgccagttggtaacctggactgtgctaatggaagttctttctgcacagaacatgtaggccaggaggaggcagggacccgggaggggggtggactttgcaggtcatctgcttagcttagtggtggccacgggttaacacgtatatagtgttactgtttgaaactccaagttttatatctgtgctgttttgatgtagaatttggggaggttcctgatgatactaccctacccgtgtatgtaagacagtctttcaacctgcagtgccagaatgtgacccacacttcagtatcttccataaagtggggggactaagaactggacaggggtgctgtggaggggggcaggccaggtgtatcttggttcctgagcagagcagagagcttaggaaggggtcgggagatctctggttcctcccaacactggtttcattttgcatggctctcttcaaacctcttgccccaggagaagcgagctttgtccaagccagctggctcgctcctttcccagatgttttaggggcctccctgaaagcttgccctcctcttaagattcagaactcctgacccagggaaagataggaggctttgtggatgggagcttttttttaaagaggaccgttctcgttctcaagtaggtagctagagagaagccccctggagcaggccctacttgtgactgtcagggaacccaggttgtgttgtaggcttttcccaggcctcccagagcagcggtgtgaaaaaatgcggtcctgggaaaagttggtctggggtgttgcttcctcgag

SEQ ID NO: 6 - DNA, 2712 bp, Mus musculus
  CDS (422)...(1903)
agggactttgcttctttttccgggctcggccgcgcagcctctccggaccctagctcgctgacgctgcgggctgcagttctcctggcggggcccgagagccgctgtctccttttctagcactcggaagggctggtgtcgctccacggtcgcgcgtggcgtctgtgccgccagctcagggctgccacccgccaagccgagagtgcgcggccagcggggccgcctgccgtgcacccttcaggatgccgatccgcccggtcggctgaacccgagcgccggcgtcttccgcgcgtggaccgcgaggctgccccgagtcggggctgcctgcatcgctccgtcccttcctgctctcctgctccgggcctcgctcgccgcgggccgcagtcggtgcgcgcaggcggcgaccgggcgtctgggacgcagcatgcaggcgcgttactcggtatcggaccccaacgccctgggagtggtaccctatttgagtgagcaaaactactaccgggcggccggcagctacggcggcatggccagccccatgggcgtctactccggccacccggagcagtacggcgccggcatgggccgctcctacgcgccctaccaccaccagcccgcggcgcccaaggacctggtgaagccgccctacagctatatagcgctcatcaccatggcgatccagaacgcgccagagaagaagatcactctgaacggcatctaccagttcatcatggaccgtttccccttctaccgcgagaacaagcagggctggcagaacagcatccgccacaacctgtcactcaatgagtgcttcgtgaaagtgccgcgcgacgacaagaagccgggcaagggcagctactggacgctcgacccggactcctacaacatgttcgagaatggcagcttcctgcggcggcggcggcgcttcaagaagaaggatgtgcccaaggacaaggaggagcgggcccacctcaaggagccgccctcgaccacggccaagggcgctccgacagggaccccggtagctgacgggcccaaggaggccgagaagaaagtcgtggttaagagcgaggcggcgtcccccgcgctgccggtcatcaccaaggtggagacgctgagccccgagggagcgctgcaggccagtccgcgcagcgcatcctccacgcccgcaggttccccagacggctcgctgccggagcaccacgccgcggcgcctaacgggctgcccggcttcagcgtggagaccatcatgacgctgcgcacgtcgcctccgggcggcgatctgagcccagcggccgcgcgcgccggcctggtggtgccaccgctggcactgccatacgccgcagcgccacccgccgcttacacgcagccgtgcgcgcagggcctggaggctgcgggctccgcgggctaccagtgcagtatgcgggctatgagtctgtacaccggggccgagcggcccgcgcacgtgtgcgttccgcccgcgctggacgaggctctgtcggaccacccgagcggccccggctccccgctcggcgccctcaacctcgcagcgggtcaggagggcgcgttgggggcctcgggtcaccaccaccagcatcacggccacctccacccgcaggcgccaccgcccgccccgcagccccctcccgcgccgcagcccgccacccaggccacctcctggtatctgaaccacggcggggacctgagccacctccccggccacacgtttgcaacccaacagcaaactttccccaacgtccgggagatgttcaactcgcaccggctaggactggacaactcgtccctcggggagtcccaggtgagcaatgcgagctgtcagctgccctatcgagctacgccgtccctctaccgccacgcagccccctactcttacgactgcaccaaatactgaggctgtccagtccgctccagccccaggaccgcaccggcttcgcctcctccatgggaaccttcttcgacggagccgcagaaagcgacggaaagcgcccctctctcagaaccaggagcagagagctccgtgcaactcgcaggtaacttatccgcagctcagtttgagatctcagcgagtccctctaagggggatgcagcccagcaaaacgaaatacagattttttttttaattccttcccctacccagatgctgcgcctgctcccttggggcttcatagattagcttatggaccaaacccatagggacccctaatgacttctgtggagattctccacgggcgcaagaggtctctccggataaggtgccttctgtaaacgagtgcggatttgtaaccaggctattttgttcttgcccagagcctttaatataatatttaaagttgtgtccactggataaggtttcgtcttgcccaactgttactgccaaattgaattcaagaaacgtgtgtgggtcttttctccccacgtcaccatgataaaataggtccctccccaaactgtaggtcttttacaaaacaagaaaataatttatttttttgttgttgttggataacgaaattaagtatcggatacttttaatttaggaagtgcatggctttgtacagtagatgccatctggggtattccaaaaacacaccaaaagactttaaaatttcaatctcacctgtgtttgtcttatgtgatctcagtgttgtatttaccttaaaataaacccgtgttgtttttctgcccaaaaaaaaaaaaaaa

SEQ ID NO: 7 - protein, 494 aa, Mus musculus
MQARYSVSDPNALGVVPYLSEQNYYRAAGSYGGMASPMGVYSGHPEQYGAGMGRSYAPYHHQPAAPKDLVKPPYSYIALITMAIQNAPEKKITLNGIYQFIMDRFPFYRENKQGWQNSIRHNLSLNECFVKVPRDDKKPGKGSYWTLDPDSYNMFENGSFLRRRRRFKKKDVPKDKEERAHLKEPPSTTAKGAPTGTPVADGPKEAEKKVVVKSEAASPALPVITKVETLSPEGALQASPRSASSTPAGSPDGSLPEHHAAAPNGLPGFSVETIMTLRTSPPGGDLSPAAARAGLVVPPLALPYAAAPPAAYTQPCAQGLEAAGSAGYQCSMRAMSLYTGAERPAHVCVPPALDEALSDHPSGPGSPLGALNLAAGQEGALGASGHHHQHHGHLHPQAPPPAPQPPPAPQPATQATSWYLNHGGDLSHLPGHTFATQQQTFPNVREMFNSHRLGLDNSSLGESQVSNASCQLPYRATPSLYRHAAPYSYDCTKY

SEQ ID NO: 8 - DNA, 3289 bp, Homo sapiens
gaattcggaggattaagttgtcagtcagcacgttgctaccttcccctctatgcactccgctgcctggctcctcggcggggagcgagggaaactcagtttgtagggtttacctctaaaacctcgataggttatccttgacgaccccgagcctggaaactccctgttgatgattaattatttgattaaataagtataacatccaggagaggccctgccattccaatccagcgcgtttgcttttgaatccattacacctgggcccccataattaggaaatctaattattcgcttcatcactcattaataagaaaaatgtcccaggatcattgctacttacaaggtctttgggagagatattttactctattaatccattctattttatatttcaaattgattttttttaacagaggaaagtggctatctttttgttttgggcatgtgggcccattcaccaaaatgtgatcataaaataaattttaataagatataactttttaaaaagttttcaagtgaagacggagtcgccgcggaggccggggcggcggggtcttagagccgacggattcctgcgctcctcgccccgattggcgccggactcctctcagctgccgggtgattggctcaaagttccgggagggggcgtggcccgaggaaagtaaaaactcgctttcagcaagaagacttttgaaacttttcccaatccctaaaagggacttggcctctttttctgggctcagcggggcagccgctcggaccccggcgcgctgaccctcggggctgccgattcgctgggggcttggagagcctcctgcgcccctcctcgcgcgggccgagggtccaccttggtccccaggccgcggcgtctccgctgggtccgcggccgcccgcctgcccgcgctgccgccgccgggtcctggagccagcgaggagcggggccggcgctgcgcttgcccggggcgcgccctccaggatgccgatccgcccggtccgctgaaagcgcgcgcccctgctcggcccgagcgacgacgaccgcgcaccctcgccccggaggctgccaggagaccggggccgcccctcccgctcccctcctctccccctctggctctctcgcgctctctcgctctcagggcccccctcgctcccccggccgcagtccgtgcgcgagggcgccggcgagccgtctcggaagcagcatgcaggcgcgctactccgtgtccgaccccaacgccctgggagtggtgccctacctgagcgagcagaattactaccgggctgcgggcagctacggcggcatggccagccccatgggcgtctattccggccacccggagcagtacagcgcggggatgggccgctcctacgcgccctaccaccaccaccagcccgcggcgcctaaggacctggtgaagccgccctacagctacatcgcgctcatcaccatggccatccagaacgcgcccgagaagaagatcaccttgaacggcatctaccagttcatcatggaccgcttccccttctaccgggagaacaagcagggctggcagaacagcatccgccacaacctctcgctcaacgagtgcttcgtcaaggtgccccgcgacgacaagaagcccggcaagggcagttactggaccctggacccggactcctacaacatgttcgagaacggcagcttcctgcggcgccggcggcgcttcaaaaagaaggacgtgtccaaggagaaggaggagcgggcccacctcaaggagccgcccccggcggcgtccaagggcgccccggccaccccccacctagcggacgcccccaaggaggccgagaagaaggtggtgatcaagagcgaggcggcgtccccggcgctgccggtcatcaccaaggtggagacgctgagccccgagagcgcgctgcagggcagcccgcgcagcgcggcctccacgcccgccggctcccccgacggttcgctgccggagcaccacgccgcggcgcccaacgggctgcctggcttcagcgtggagaacatcatgaccctgcgaacgtcgccgccgggcggagagctgagcccgggggccggacgcgcgggcctggtggtgccgccgctggcgctgccatacgccgccgcgccgcccgccgcctacggccagccgtgcgctcagggcctggaggccggggccgccgggggctaccagtgcagcatgcgagcgatgagcctgtacaccggggccgagcggccggcgcacatgtgcgtcccgcccgccctggacgaggccctctcggaccacccgagcggccccacgtcgcccctgagcgctctcaacctcgccgccggccaggagggcgcgctcgccgccacgggccaccaccaccagcaccacggccaccaccacccgcaggcgccgccgcccccgccggctccccagccccagccgacgccgcagcccggggccgccgcggcgcaggcggcctcctggtatctcaaccacagcggggacctgaaccacctccccggccacacgttcgcggcccagcagcaaactttccccaacgtgcgggagatgttcaactcccaccggctggggattgagaactcgaccctcggggagtcccaggtgagtggcaatgccagctgccagctgccctacagatccacgccgcctctctatcgccacgcagccccctactcctacgactgcacgaaatactgacgtgtcccgggacctcccctccccggcccgctccggcttcgcttcccagccccgacccaaccagacaattaaggggctgcagagacgcaaaaaagaaacaaaacatgtccaccaaccttttctcagacccgggagcagagagcgggcacgctagcccccagccgtctgtgaagagcgcaggtaactttaattcgccgccccgtttctgggatcccaggaaacccctccaaagggacgcagcccaacaaaatgagtattggtcttaaaatccccctcccctaccaggacggctgtgctgtgctcgacctgagctttcaaaagttaagttatggacccaaatcccatagcgagcccctagtgactttctgtaggggtccccataggtgtatgggggtctctatagataatatatgtgctgtgtgtaattttaaatttctccaaccgtgctgtacaaatgtgtggatttgtaatcaggctattttgttgttgttgttgttgttcagagccattaatataatatttaaagttgagttcactggataagtttttcatcttgcccaaccatttctaactgccaaattgaattc

SEQ ID NO: 9 - DNA, 1506 bp, Homo sapiens
  CDS (1)...(1503)
atgcaggcgcgctactccgtgtccgaccccaacgccctgggagtggtgccctacctgagcgagcagaattactaccgggctgcgggcagctacggcggcatggccagccccatgggcgtctattccggccacccggagcagtacagcgcggggatgggccgctcctacgcgccctaccaccaccaccagcccgcggcgcctaaggacctggtgaagccgccctacagctacatcgcgctcatcaccatggccatccagaacgcgcccgagaagaagatcaccttgaacggcatctaccagttcatcatggaccgcttccccttctaccgggagaacaagcagggctggcagaacagcatccgccacaacctctcgctcaacgagtgcttcgtcaaggtgccccgcgacgacaagaagcccggcaagggcagttactggaccctggacccggactcctacaacatgttcgagaacggcagcttcctgcggcgccggcggcgcttcaaaaagaaggacgtgtccaaggagaaggaggagcgggcccacctcaaggagccgcccccggcggcgtccaagggcgccccggccaccccccacctagcggacgcccccaaggaggccgagaagaaggtggtgatcaagagcgaggcggcgtccccggcgctgccggtcatcaccaaggtggagacgctgagccccgagagcgcgctgcagggcagcccgcgcagcgcggcctccacgcccgccggctcccccgacggttcgctgccggagcaccacgccgcggcgcccaacgggctgcctggcttcagcgtggagaacatcatgaccctgcgaacgtcgccgccgggcggagagctgagcccgggggccggacgcgcgggcctggtggtgccgccgctggcgctgccatacgccgccgcgccgcccgccgcctacggccagccgtgcgctcagggcctggaggccggggccgccgggggctaccagtgcagcatgcgagcgatgagcctgtacaccggggccgagcggccggcgcacatgtgcgtcccgcccgccctggacgaggccctctcggaccacccgagcggccccacgtcgcccctgagcgctctcaacctcgccgccggccaggagggcgcgctcgccgccacgggccaccaccaccagcaccacggccaccaccacccgcaggcgccgccgcccccgccggctccccagccccagccgacgccgcagcccggggccgccgcggcgcaggcggcctcctggtatctcaaccacagcggggacctgaaccacctccccggccacacgttcgcggcccagcagcaaactttccccaacgtgcgggagatgttcaactcccaccggctggggattgagaactcgaccctcggggagtcccaggtgagtggcaatgccagctgccagctgccctacagatccacgccgcctctctatcgccacgcagccccctactcctacgactgcacgaaatactga

SEQ ID NO: 10 - protein, 501 aa, Homo sapiens; identical to SEQ ID NO: 2

SEQ ID NO: 11 - DNA, 327 bp, Homo sapiens
ttttttttacattttcgtcttctgttcttgtgattggaaataagtggcacgccccattgccttctagtcgcctccccgaagcgaagaggccgaagcgaagaggcctggtgggttgtctcaacatccttttgctgagaatcgaatacgcagccgatgaacagccaggaagggtgcaaggaaaccttgaacggcatctaccagttcatcatggaccgcttccccttctaccgggagaacaagcagggctggcagaacagcatccgccacaacctctcgctcaacgagtgcttcgtcaaggtgccccgcgacgacaagaagcccggcaag

SEQ ID NO: 12 - DNA, 147 bp, Homo sapiens
ccgtctgagaatcgaatacgcagccgatgaacagccaggaagggtgcaaggaaaccttgaacggcatctaccagttcatcatggaccgcttccccttctaccgggagaacaagcagggctggcagaacagcatccgccacaacctct

SEQ ID NO: 13 - DNA, 878 bp, Homo sapiens
gtctctctcaccttttctgtcttgatgagacgaatttctttcccctccccttttcctttctttggggcgggggagggtggataatatattgggcgactcgatttaggtgtttgtttgtttgtttgtttgtttccccagatgacattggtttaaaccgggacacccttgtgaatacaaacgtaggcagcaactgccattttggaatttattttttcatagtccttagctattttaggttttgctgtgataaagctgtttctctctctctctctctctcacacacacacacacacccctcgtaaaagcagagtaaataatattcctccaggaagcctacaggctgaggagtgtttcttgatcaatagtttgcatttccagtaaaatcgtgacacgaactcagtgtgcctgtcatgcgctgcaggagaagggcactttttgctagtccttttttttttttaagctagatgcggaaatactagcttattaaaaataataaagtcatggtgggagtttagggttggggcagaaagctcaaatcatttgcctgtgagctgagaactgggcagctttattttactttgtttcaaagaaagaagaaaaaggatcaggttagaaaaagagcccagaatactcataaaaacaatgtttcagaagtggaatattcaaggtaaaggaacctgatttgtagcttccctttggctttgaattgatcaggagacaaagataatgcatctacattttcgtcttctgttcttttattggaaataagtggcacgccccattgccttctagtcgcctccccgaagcgaagaggccgaagcgaagaggcctggtgggttgtctcaacatccttttgctgagaatcgaatacgcagccg

SEQ ID NO: 14 - DNA, 22 bp, Artificial Sequence
  primer for PCR
ccattgccttctagtcgcctcc

SEQ ID NO: 15 - DNA, 22 bp, Artificial Sequence
  primer for PCR
cgttggggtcggacacggagta

SEQ ID NO: 16 - DNA, 27 bp, Artificial Sequence
  primer for PCR
ggtacctacgcagccgatgaacagcca

SEQ ID NO: 17 - DNA, 26 bp, Artificial Sequence
  primer for PCR
gctagcgctgcttccgagacggctcg

SEQ ID NO: 18 - DNA, 27 bp, Artificial Sequence
  primer for PCR
ggtaccccccgagcctggaaactccct

SEQ ID NO: 19 - DNA, 25 bp, Artificial Sequence
  primer for PCR
atgaacagccaggaagggtgcaagg

SEQ ID NO: 20 - DNA, 24 bp, Artificial Sequence
  primer for PCR
acagccaggaagggtgcaaggaaa

SEQ ID NO: 21 - DNA, 25 bp, Artificial Sequence
  primer for PCR
gaagctgccgttctcgaacatgttg

SEQ ID NO: 22 - DNA, 25 bp, Artificial Sequence
  primer for PCR
gtaggagtccgggtccagggtccag

SEQ ID NO: 23 - DNA, 26 bp, Artificial Sequence
  primer for PCR
gcgttcggctcactgacttacaaggt

SEQ ID NO: 24 - DNA, 31 bp, Artificial Sequence
  primer for PCR
ggaagtgtctctctcaccttttctgtcttga

(19) United States
(12) Patent Application Publication
(10) Pub. No.: US 2002/0090707 A1
(43) Pub. Date: Jul. 11, 2002

(54) Promoter sequences

(76) Inventors: Sven Enerback, Molndal (SE); Katarina Krook, Stockholm (SE); Lena Rondahl, Bromma (SE); Wyeth Wasserman, Stockholm (SE)

Correspondence Address: JANIS K. FRASER, Ph.D., Fish & Richardson P.C., 225 Franklin Street, Boston, MA, 02110-2804, US

(21) Appl. No.: 09/963,285

(22) Filed: Sep. 26, 2001

Related U.S. Application Data
(60) Provisional application No. 60/238,897, filed on Oct. 10, 2000.

(30) Foreign Application Priority Data
Sep. 26, 2000 (SE) 0003435-5
Nov. 9, 2000 (SE) 0004102-0

(51) Int. Cl. C12Q 1/68; C07H 21/04; C12P 19/34; C12N 9/22; C12P 21/02; C12N 5/06

(52) U.S. Cl. 435/199; 435/6; 435/69.1; 435/325; 435/320.1; 435/91.2; 536/23.2

(57) ABSTRACT

The present invention relates an isolated promoter region of the mammalian transcription factor FOXC2. The invention also relates to screening methods for agents modulating the expression of FOXC2 and thereby being potentially useful for the treatment of medical conditions related to obesity. The invention further relates to a previously unknown variant of the human FOXC2 gene, derived via the use of an alternative promoter, which produces an additional exon that generates a distinct open reading frame via splicing. The alternative gene encodes a variant of the FOXC2 transcription factor, which is lacking a part of the DNA-binding domain and consequently has a potential regulatory function.

### CROSS REFERENCE TO RELATED APPLICATIONS

This application claims priority from Swedish Patent Application No. 0003435-5, filed Sep. 26, 2000, U.S. Provisional Patent Application Serial No. 60/238,897, filed Oct. 10, 2000, and Swedish Patent Application No. 0004102-0, filed Nov. 9, 2000. These applications are incorporated herein by reference in their entirety.

### TECHNICAL FIELD

The present invention relates an isolated promoter region of the mammalian transcription factor FOXC2. The invention also relates to screening methods for agents modulating the expression of FOXC2 and thereby being potentially useful for the treatment of medical conditions related to obesity. The invention further relates to a previously unknown variant of the human FOXC2 gene, derived via the use of an alternative promoter, which produces an additional exon that generates a distinct open reading frame via splicing. The alternative gene encodes a variant of the FOXC2 transcription factor, which is lacking a part of the DNA-binding domain and consequently has a potential regulatory function.

### BACKGROUND

More than half of the men and women in the United States, 30 years of age and older, are now considered overweight, and nearly one-quarter are clinically obese. This high prevalence has led to increases in the medical conditions that often accompany obesity, especially non-insulin dependent diabetes mellitus (NIDDM), hypertension, cardiovascular disorders, and certain cancers. Obesity results from a chronic imbalance between energy intake (feeding) and energy expenditure. To better understand the mechanisms that lead to obesity and to develop strategies in certain patient populations to control obesity, there is a need to develop a better underlying knowledge of the molecular events that regulate the differentiation of preadipocytes and stem cells to adipocytes, the major component of adipose tissue.

The helix-loop-helix (HLH) family of transcriptional regulatory proteins are key players in a wide array of developmental processes (for a review, see Massari & Murre (2000) Mol. Cell. Biol. 20: 429-440). Over 240 HLH proteins have been identified to date in organisms ranging from the yeast Saccharomyces cerevisiae to humans. Studies in Xenopus laevis, Drosophila melanogaster, and mice have convincingly demonstrated that HLH proteins are intimately involved in developmental events such as cellular differentiation, lineage commitment, and sex determination. In multicellular organisms, HLH factors are required for a multitude of important developmental processes, including neurogenesis, myogenesis, hematopoiesis, and pancreatic development.

The winged helix/forkhead class of transcription factors is characterized by a 100-amino acid, monomeric DNA-binding domain. X-ray crystallography of the forkhead domain from HNF-3γ has revealed a three-dimensional structure, the “winged helix”, in which two loops (wings) are connected on the C-terminal side of the helix-loop-helix (for reviews, see Brennan, R. G. (1993) Cell 74: 773-776; and Lai, E. et al. (1993) Proc. Natl. Acad. Sci. U.S.A. 90: 10421-10423).

The isolation of the mouse mesenchyme forkhead-1 (MFH-1) and the corresponding human (FKHL14) chromosomal genes is disclosed by Miura, N. et al. (1993) FEBS letters 326: 171-176; and (1997) Genomics 41: 489-492. The nucleotide sequences of the mouse MFH-1 gene and the human FKHL14 gene have been deposited with the EMBL/GenBank Data Libraries under accession Nos. Y08222 (SEQ ID NO:5) and Y08223 (SEQ ID NO:8), respectively. A corresponding gene has been identified in Gallus gallus (GenBank accession numbers U37273 and U95823).

The International Patent Application WO 98/54216 discloses a gene encoding a Forkhead-Related Activator (FREAC)-11 (also known as S12), which is identical with the polypeptide encoded by the human FKHL14 gene disclosed by Miura, supra. This transcription factor is expressed in adipose tissue and involved in lipid metabolism and adipocyte differentiation (cf. Swedish patent application No. 0000531-4, filed Feb. 18, 2000).

The nomenclature for the winged helix/forkhead transcription factors has been standardized and Fox (Forkhead Box) has been adopted as the unified symbol (Kaestner et al. (2000) Genes & Development 14: 142-146; see also htpp.//www.biology.pomona.edu/fox). It has been agreed that the genes previously designated MFH-1 and FKHL14 (as well as FREAC-11 and S12) should be designated FOXC2.

### BRIEF DESCRIPTION OF THE DRAWINGS

FIG. 1 shows the general structure of the human FOXC2 gene.

FIG. 2 illustrates the results from phylogenetic footprinting experiments. Shown is the fraction conserved (1.0=100%) between mouse FoxC2 and human FOXC2 sequences in the alignment generated with Clustal. Solid (bold) line indicates the fraction of the human sequence which is identical to the mouse within a 200 bp “window” over the human sequence in the alignment. The weak (dotted) line is set to −0.05 when the sliding window contains human exon sequence and to −0.1 when the window is entirely composed of exon sequence. Regions containing local maxima or exceeding a conservation fraction of 0.7 are likely to be functional and are classified as “predicted regulatory regions”.

FIG. 3 illustrates the predicted “enhancer” region in the human FOXC2 gene (HUMAN: nucleotides 200-475 of SEQ ID NO:1; MOUSE: nucleotides 174-461 of SEQ ID NO:5). Underlined sequences indicate likely transcription factor binding sites. Boxed sequence indicates exon sequence.

Splice=sequence predicted as splice site in the alternatively spliced gene;

E-box-like=sequence resembling the “E-box” motif CANNTG known as a target for DNA binding proteins containing a helix-loop-helix domain (often associated with the activation of cell-type specific gene transcription during tissue differentiation; see Massari & Murre (2000) Mol. Cell. Biol. 20: 429-440)

Forkhead-like=sequence resembling binding site for the winged helix/forkhead class of transcription factors;

Ets-like=sequence resembling consensus binding site for ETS-domain transcription factor family (see Sharrocks et al. (1997) Int. J. Biochem. Cell Biol. 29, 1371-1387).

FIG. 4 illustrates the predicted “promoter” region in the human FOXC2 gene (HUMAN: nucleotides 1251-1763 of SEQ ID NO:1; MOUSE: nucleotides 1126-1662 of SEQ ID NO:5). Underlined sequence indicates exon sequences. Boxed sequences indicate conserved block (potential transcription factor binding sites).

### DESCRIPTION OF THE INVENTION

According to the present invention, the partially known sequence (SEQ ID NO: 8) of human FOXC2 gene has been extended. In the previously unknown region of the gene, differentially conserved regions, consistent with regulatory function, have been identified. Further, an alternative transcript has been identified, which includes the use of at least two exons. The putative regulatory enhancer is immediately adjacent to the newly discovered alternative exon, suggesting that it may play a role in the alternative selection of transcript classes.

Modulation of the FOXC2 regulation is expected to have therapeutic value in type II diabetes; obesity, hypercholesterolemia, and other cardiovascular diseases or dyslipidemias.

Consequently, in a first aspect this invention provides an isolated human FOXC2 promoter region comprising a sequence selected from:

(a) the nucleotide sequence set forth as positions 1250 to 2235, such as positions 1250 to 1749 or positions 1692 to 1703, in SEQ ID NO:1, or a fragment thereof exhibiting FOXC2 promoter activity;

(b) the complementary strand of (a); and

(c) nucleotide sequences capable of hybridizing, under stringent hybridization conditions, to a nucleotide sequence as defined in (a) or (b).

An “isolated” nucleic acid is a nucleic acid molecule the structure of which is not identical to that of a naturally occurring nucleic acid or to that of any fragment of a naturally occurring genomic nucleic acid spanning more than one gene.

“Stringent” hybridization conditions are hybridization in 6× SSC at 45° C., followed by one or more washes in 0.2× SSC, 0.1% SDS at 65° C.

“Promoter region” refers to a region of DNA that functions to control the transcription of one or more coding sequences, and is structurally identified by the presence of a binding site for DNA-dependent RNA polymerase and of other DNA sequences on the same molecule which interact to regulate promoter function.

Another aspect of the invention is a recombinant construct comprising the human FOXC2 promoter region as defined above. In the said recombinant construct, the human FOXC2 promoter region can be operably linked to a gene encoding a detectable product, such as the human FOXC2 gene, or a reporter gene. The term “operably linked” as used herein means functionally fusing a promoter with a structural gene in the proper frame to express the structural gene under control of the promoter. As used herein, the term “reporter gene” means a gene encoding a gene product that can be identified using simple, inexpensive methods or reagents and that can be operably linked to the human FOXC2 promoter region or an active fragment thereof. Reporter genes such as, for example, a luciferase, β-galactosidase, alkaline phosphatase, or green fluorescent protein reporter gene, can be used to determine transcriptional activity in screening assays according to the invention (see, for example, Goeddel (ed.), Methods Enzymol., Vol. 185, San Diego: Academic Press, Inc. (1990); see also Sambrook, supra).

The invention also provides a vector comprising the recombinant construct as defined above, as well as a host cell stably transformed with such a vector, or generally with the recombinant construct according to the invention. The term “vector” refers to any carrier of exogenous DNA that is useful for transferring the DNA to a host cell for replication and/or appropriate expression of the exogenous DNA by the host cell.

In another aspect, the invention provides a method for identification of an agent regulating FOXC2 promoter activity, said method comprising the steps: (i) contacting a candidate agent with a human FOXC2 promoter region as defined above; and (ii) determining whether said candidate agent modulates expression of the FOXC2 gene, such modulation being indicative for an agent capable of regulating FOXC2 promoter activity. As used herein, the term “agent” means a biological or chemical compound such as a simple or complex organic molecule, a peptide, a protein or an oligonucleotide.

A transfection assay can be a particularly useful screening assay for identifying an effective agent modulating and/or regulating FOXC2 promoter activity. In a transfection assay, a nucleic acid containing a gene, e.g. a reporter gene, operably linked to a human FOXC2 promoter or an active fragment thereof, is transfected into the desired cell type. A test level of reporter gene expression is assayed in the presence of a candidate agent and compared to a control level of expression. An effective agent is identified as an agent that results in a test level of expression that is different than a control level of reporter gene expression, which is the level of expression determined in the absence of the agent. Methods for transfecting cells and a variety of convenient reporter genes are well known in the art (see, for example, Goeddel (ed.), Methods Enzymol., Vol. 185, San Diego: Academic Press, Inc. (1990); see also Sambrook, supra). Consequently, the said method could e.g. comprising assaying reporter gene expression in a host cell, stably transformed with a recombinant construct comprising the human FOXC2 promoter, in the presence and absence of a candidate agent, wherein an effect on the test level of expression as compared to control level of expression is indicative of an agent capable of regulating FOXC2 promoter activity.

Methods for identification of polypeptides regulating FOXC2 promoter activity could include various techniques known in the art, such as the yeast one-hybrid system (see: Li & Herskowitz (1993) Science 262, 1870-1874) to identify proteins binding specific sequences from the FOXC2 regulatory region, biochemical purification of proteins which bind to the regulatory region, the use of a “southwestern” cloning strategy (see e.g. Hai et al. (1989) Genes & Development 3: 2083-2090) in which a pool of bacteria infected with a “phage library” are induced to express the encoded protein and probed with radioactive DNA sequences from the FOXC2 regulatory regions to identify binding proteins.

In a further aspect, the invention provides an isolated human FOXC2 enhancer region comprising a sequence selected from:

(a) the nucleotide sequence set forth as positions 216 to 475, such as positions 223 to 231, positions 359 to 375, positions 378 to 402, or positions 403 to 423, in SEQ ID NO:1, or a fragment thereof exhibiting FOXC2 enhancer activity;

(b) the complementary strand of (a); and

(c) nucleotide sequences capable of hybridizing, under stringent hybridization conditions, to a nucleotide sequence as defined in (a) or (b).

“Enhancer region” refers to a region of DNA that functions to control the transcription of one or more coding sequences.

As described above for the human FOXC2 promoter region, the invention further provides a recombinant construct comprising a human FOXC2 enhancer region, a vector comprising the said recombinant construct, as well as a host cell stably transformed with said vector or with said recombinant construct.

Further, the invention provides a method for identification of an agent regulating FOXC2 enhancer activity, said method comprising the steps: (i) contacting a candidate agent with the human FOXC2 enhancer region as defined above; and (ii) determining whether said candidate agent modulates expression of the FOXC2 gene, such modulation being indicative for an agent capable of regulating FOXC2 enhancer activity. It will be understood by the skilled person that known steps are available for performing such a method. For instance, a “panel” of constructs which include a variety of mutations and deletions can be used in order to associate a response with a specific alteration of a single base or subsegment of the regulatory apparatus. A simple panel might include: enhancer plus promoter, promoter only, enhancer plus a “minimal” promoter from a distinct gene. As mentioned above, a transfection assay, using a host cell stably transformed with a suitable recombinant construct, can be a particularly useful screening assay for identifying an effective agent.

In yet a further aspect, the invention provides a method for identification of an agent capable of regulating a mammalian FOXC2 promoter activity, said method comprising the steps (i) contacting a candidate agent with a murine FoxC2 promoter nucleotide sequence shown as positions 216 to 2235, such as positions 216 to 475 or positions 1250 to 2235, in SEQ ID NO:5; and (ii) determining whether said candidate agent modulates expression of a mammalian FOXC2 gene, such modulation being indicative for an agent capable of regulating mammalian FOXC2 promoter activity.

In another important aspect, the invention provides an isolated nucleic acid molecule selected from:

(a) nucleic acid molecules comprising a nucleotide sequence as shown in SEQ ID NO:3;

(b) nucleic acid molecules comprising a nucleotide sequence capable of hybridizing, under stringent hybridization conditions, to a nucleotide sequence complementary the polypeptide coding region of a nucleic acid molecule as defined in (a) and which codes for a variant form of the FOXC2 transcription factor; and

(c) nucleic acid molecules comprising a nucleic acid sequence which is degenerate as a result of the genetic code to a nucleotide sequence as defined in (a) or (b) and which codes for a variant form of the FOXC2 transcription factor.

In a preferred form of the invention, the said nucleic acid molecule has a nucleotide sequence identical with SEQ ID NO:3 of the Sequence Listing. However, the nucleic acid molecule according to the invention is not to be limited strictly to the sequence shown as SEQ ID NO:3. Rather the invention encompasses nucleic acid molecules carrying modifications like substitutions, small deletions, insertions or inversions, which nevertheless encode proteins having substantially the biochemical activity of the FOXC2 polypeptide according to the invention. Included in the invention are consequently nucleic acid molecules, the nucleotide sequence of which is at least 90% homologous, preferably at least 95% homologous, with the nucleotide sequence shown as SEQ ID NO:3 in the Sequence Listing.

Included in the invention is also a nucleic acid molecule which nucleotide sequence is degenerate, because of the genetic code, to the nucleotide sequence shown as SEQ ID NO:3. A sequential grouping of three nucleotides, a “codon”, codes for one amino acid. Since there are 64 possible codons, but only 20 natural amino acids, most amino acids are coded for by more than one codon. This natural “degeneracy”, or “redundancy”, of the genetic code is well known in the art. It will thus be appreciated that the nucleotide sequence shown in the Sequence Listing is only an example within a large but definite group of sequences which will encode the variant FOXC2 polypeptide.

The invention includes an isolated polypeptide encoded by the nucleic acid as defined above. In a preferred form, the said polypeptide has an amino acid sequence according to SEQ ID NO:4 of the Sequence Listing. However, the polypeptide according to the invention is not to be limited strictly to a polypeptide with an amino acid sequence identical with SEQ ID NO:4 in the Sequence Listing. Rather the invention encompasses polypeptides carrying modifications like substitutions, small deletions, insertions or inversions, which polypeptides nevertheless have substantially the biological activities of the variant FOXC2 polypeptide. In one embodiment, the polypeptide includes an amino acid sequence that is at least about 70%, 75%, 80%, 85%, 90%, 95%, 98% or more identical to the amino acid sequence of SEQ ID NO:4.

An “isolated” polypeptide is substantially free of other contaminating proteins from the cell or tissue source from which the protein is derived, or substantially free from chemical precursors or other chemicals when chemically synthesized.

A further aspect of the invention is a vector harboring the nucleic acid molecule according to the invention. The said vector can e.g. be a replicable expression vector, which carries and is capable of mediating the expression of a DNA molecule according to the invention. In the present context the term “replicable” means that the vector is able to replicate in a given type of host cell into which is has been introduced. Examples of vectors are viruses such as bacteriophages, cosmids, plasmids and other recombination vectors. Nucleic acid molecules are inserted into vector genomes by methods well known in the art.

Included in the invention is also a cultured host cell harboring a vector according to the invention. Such a host cell can be a prokaryotic cell, a unicellular eukaryotic cell or a cell derived from a multicellular organism. The host cell can thus e.g. be a bacterial cell such as an E. coli cell; a cell from yeast such as Saccharomyces cervisiae or Pichia pastoris, or a mammalian cell. The methods employed to effect introduction of the vector into the host cell are standard methods well known to a person familiar with recombinant DNA methods.

In yet another aspect, the invention includes a method for identifying an agent capable of regulating expression of the nucleic acid molecule as defined above, said method comprising the steps (i) contacting a candidate agent with the said nucleic acid molecule; and (ii) determining whether said candidate agent modulates expression of the said nucleic acid molecule.

In another aspect the invention provides an antisense oligonucleotide having a sequence capable of specifically hybridizing to RNA transcribed by the alternatively spliced nucleic acid molecule shown as SEQ ID NO:3, so as to prevent translation of the said RNA. Antisense nucleic acids (preferably 10 to 20 base-pair oligonucleotides) capable of specifically binding to control sequences for the alternatively spliced FOXC2 gene are introduced into cells, e.g. by a viral vector or colloidal dispersion system such as a liposome. The antisense nucleic acid binds to the target nucleotide sequence in the cell and prevents transcription and/or translation of the target sequence. Phosphorothioate and methylphosphonate antisense oligonucleotides are specifically contemplated for therapeutic use by the invention. Suppression of expression of the alternatively spliced FOXC2 gene, at either the transcriptional or translational level, is useful to generate cellular or animal models for diseases/conditions related to lipid metabolism.

In yet another aspect, the invention provides a method for the identification of polypeptides which bind to nucleotide sequences involved in the biological pathway regulating lipid metabolism and/or adipocyte differentiation, comprising the steps of:

(a) transfecting a host cell line with a human FOXC2 nucleotide sequence linked to a reporter gene, such as a gene encoding Green Fluorescent Protein (GFP) (for a review, see e.g. Galbraith et al. (1999) Methods in Cell Biology 58: 315-341);

(b) transfecting the said host cell line with a variety of human cDNA sequences, e.g. sequences included in a cDNA library;

(c) identifying and isolating cells, e.g. by FACS cells sorting, having an altered level of expression of the said reporter gene, which is indicative that the polypeptide encoded by the added cDNA up- or downregulates at least one gene involved in the biological pathway regulating lipid metabolism and/or adipocyte differentiation;

(d) recovering cDNA from the cells isolated in step (c), by standard procedures, e.g. PCR or a CRE-LOX mediated procedure (see e.g. Sauer (1998) Methods 14: 381-392); and

(e) identifying the polypeptide expressed by the cDNA recovered in step (d), e.g. by sequencing the cDNA and comparing the obtained sequence against sequence databases.

In yet another aspect, the invention includes a nucleic acid comprising a nucleotide sequence selected from the group consisting of nucleotides 1692 to 1703 of SEQ ID NO:1, nucleotides 223 to 231 of SEQ ID NO:1, nucleotides 359 to 375 of SEQ ID NO:1, nucleotides 378 to 402 of SEQ ID NO:1, and nucleotides 403 to 423 in SEQ ID NO:1, operably linked to a heterologous coding sequence. The nucleotide sequence can optionally comprise any of the promoter or enhancer sequences described herein. A “heterologous coding sequence” is any coding sequence other than one that encodes a naturally occurring FOXC2 protein.

Throughout this description the terms “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2^{nd} Ed., Cold Spring Harbor Laboratory Press, Cold Spring Harbor, N.Y. 1989.

### EXAMPLES

### Example 1

### Computational Identification of FOXC2 Genomic Sequences

The sequences present in the GenBank database (http://www.ncbi.nlm.nih.gov) were screened for sequence similarity to the human FOXC2 cDNA sequence (GenBank accession number NM_{—}00521 (SEQ ID NO:9)). The BLAST algorithm (Altschul et al. (1997) Nucleic Acids Res. 25:3389-3402) was used for determining sequence identity. Software for performing BLAST analyses is publicly available through the National Center for Biotechnology Information (http.//www.ncbi.nlm.nih.gov). A working draft genomic sequence in 25 unordered pieces, from the Homo sapiens chromosome 16 clone RP11-463O9 (GenBank accession number AC009108; Version 6; GI:7689930; released May 4, 2000), was selected for further studies.

Regions in sequence AC009108 matching portions of the FOXC2 cDNA sequence NM_{—}005251 were combined using the PHRAP software, developed at the University of Washington (http.//www.genome.washington.edu/UWGC/analysistools/phrap.htm). Two contigs of 9780 bp (positions 116445 to 126224 in GenBank AC009108.6) and 3784 bp (positions 42927 to 46710 in GenBank AC0091108.6), respectively, were assembled to generate a human FOXC2 genomic fragment of 13451 bp.

The ClustalW multiple sequence alignment program, version 1.8 (Thompson et al. (1994) Nucleic Acids Research 22: 4673-4680), was then used to identify the human FOXC2 extended genomic DNA sequence of 6458 bp (SEQ ID NO:1) by comparison with the mouse cDNA sequence X74040 (SEQ ID NO:6). First, a 6459 bp sequence, corresponding to positions 1500-7958 in the 13451 bp sequence, was selected. Positions 1-2285 in this 6459 bp sequence corresponded to 44426-46710 in AC009108.6, while positions 2151-6459 corresponded to positions 126224-121916 (reverse complement taken) in AC009108.6. The overlap of positions 2151-2285 allowed for the contigs to be joined by the assembly program. The G residue in position 2655 was considered to be a sequencing error and was removed, which resulted in the 6458 bp sequence set forth as SEQ ID NO:1. The open reading frame in SEQ ID NO:1 encodes a polypeptide (SEQ ID NO:2) identical with the known human FOXC2 polypeptide shown as SEQ ID NO:10.

### Example 2

### Identification of Potential Regulatory Sequences in the Human and Mouse FOXC2 Genomic Sequences

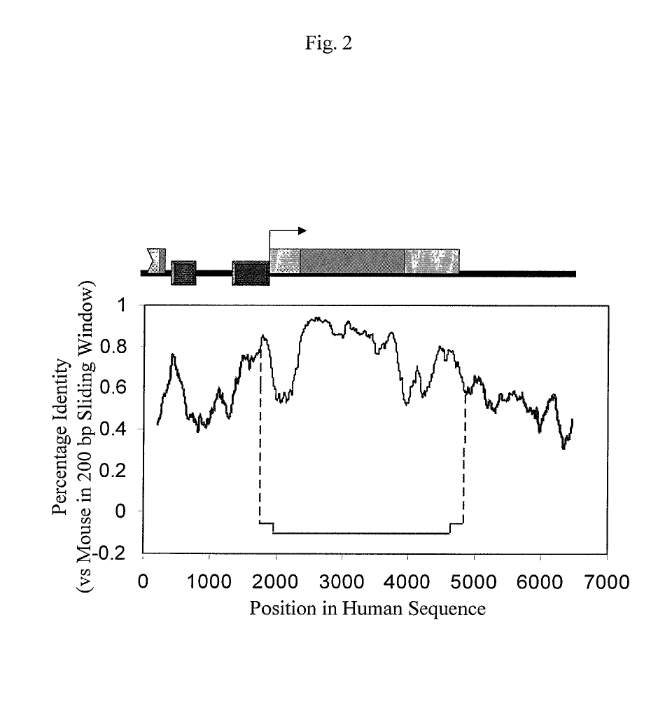

In phylogenetic footprinting (for a review, see Duret & Bucher (1997) Current Opinion in Structural Biology 7(3): 399-406) sequences are aligned and a regional sequence identity is determined for each window of a fixed, arbitrary length. This allows the identification of potential regulatory regions in genomic sequences. Non-exon sequences that are conserved over the course of evolution are likely to perform regulatory roles. Phylogenetic footprinting was performed as described in Wasserman & Fickett (1998) J. Mol. Biol. 278, 167-181, based on an alignment generated with the ClustalW multiple sequence alignment program, version 1.8 (Thompson et al. (1994) Nucleic Acids Research 22: 4673-4680), with default parameters adjusted to a gap opening penalty of 20 and a gap extension penalty of 0.2. The human (SEQ ID NO:1) and mouse (SEQ ID NO:5) genomic sequences were aligned. Percentage identity was plotted for each contiguous 200 bp segment of the human gene to identify segments differentially conserved (in comparison to adjoining sequences) (FIG. 2).

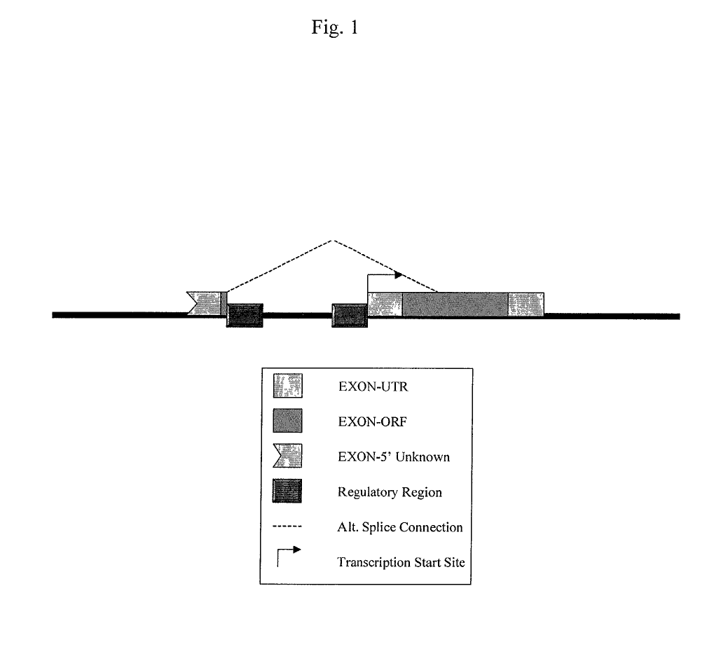

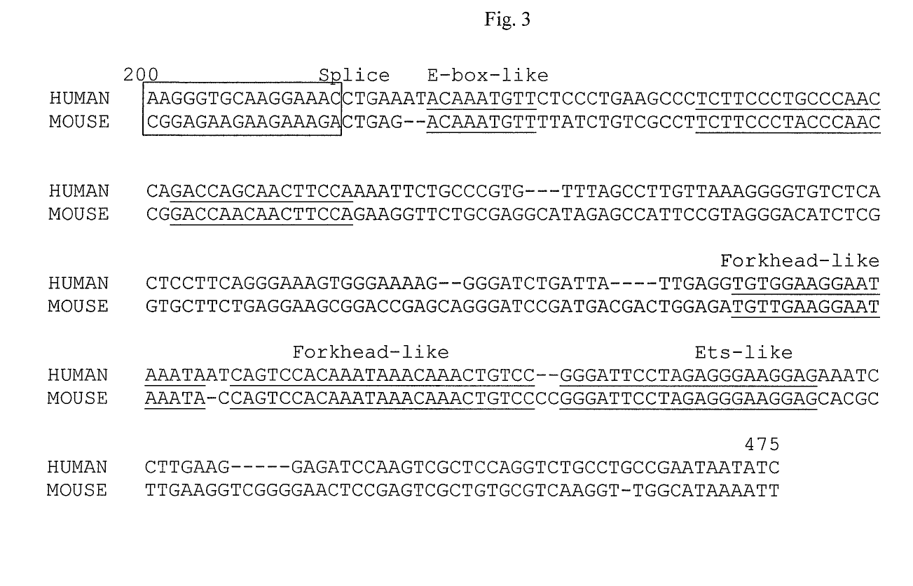

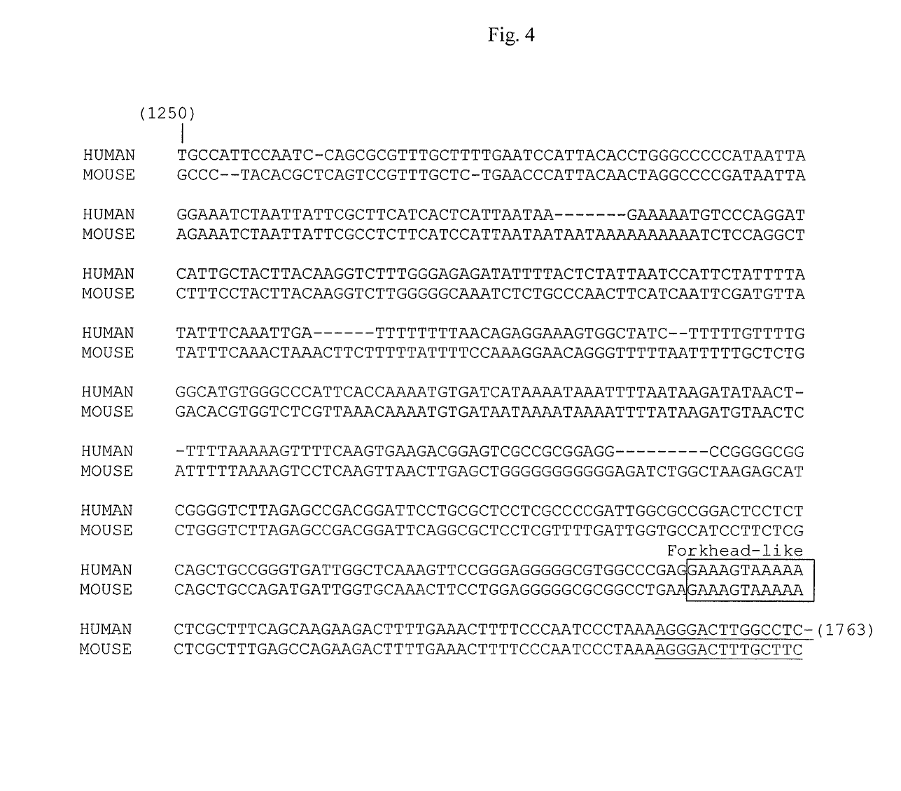

In addition to segments of the published exon sequence, two differentially conserved regions or “footprints” were identified in the human gene. Both of these regions are local maxima and contain segments which exceed 70% nucleotide identity between the human and mouse genomic sequences. One region, shown as positions 1250 to 2235, in particular positions 1250 to 1749, in SEQ ID NO:1, immediately adjacent to the published exon region, is likely to contain the transcription start site and proximal promoter regulatory sequences (FIG. 4). Another region, shown as positions 216 to 475 in SEQ ID NO:1, approximately 1700 bp distal from the transcription start site, is likely to function as some form of regulatory region (either enhancer or repressor) (FIG. 3). (A schematic overview of the extended FOXC2 gene is shown in FIG. 1).

Further analysis of these regulatory regions identified short segments of higher conservation between the mouse and human genes, suggesting that these specific segments function as transcription factor binding sites. The TRANSFAC transcription factor database (http://transfac.gbf.de) (see Wingender et al. (2000) Nucleic Acids Research 28(1): 316-319) was screened for matches to known transcription factors. Consensus sites (identifiers R05066; R05067; R05068; and R05069) were found to match sequences conserved between the human FOXC2 and mouse FoxC2 genes. This suggests the presence of multiple forkhead-like binding sites in the distal regulatory enhancer, and potential auto-regulation of FOXC2 by its protein product.

The same analysis was performed with reference to 200 bp contiguous segments of the mouse FoxC2 genomic sequence (SEQ ID NO:5). The following conserved regions were identified: 190 to 420; 1070 to 1645; and 5580 to 5875. They correlate to the regions indicated above for the human sequence and should be considered orthologous regions.

### Example 3

### Identification of an Alternative Human FOXC2 cDNA Sequence

BLASTN screening of the dbEST database from GenBank, using the human FOXC2 cDNA (SEQ ID NO:9) as a query sequence, revealed several ESTs overlapping containing portions of the available cDNA. A specialized tool, est_genome (http://www.sanger.ac.uk), for the prediction of exon boundaries using ESTs was applied to compare the EST sequences to the genomic sequences (See Mott, R. (1997) Computer Applications in the Biosciences 13(4): 477-478). Two classes of ESTs were observed: sequences extending into the 3′-untranslated region and sequences revealing an alternative first exon spliced to a junction internal to the previously described first exon.

Specifically, it was found that the nucleotides in positions 33 to 182 in the EST with accession no. AW271272 (SEQ ID NO:11) were identical to positions 66 to 215 in the extended FOXC2 genomic sequence (SEQ ID NO:1), and that positions 183 to 327 in SEQ ID NO:11 were identical to positions 2516 to 2660 in SEQ ID NO:1. Similarly, positions 5 to 55 in the EST with accession no. AW793237 (SEQ ID NO:12) were identical to positions 165 to 215 in the extended FOXC2 genomic sequence (SEQ ID NO:1), and positions 56 to 157 in SEQ ID NO:12 were identical to positions 2516 to 2607 in SEQ ID NO:1. These results revealed an alternative splicing pattern in the human FOXC2 gene. According to this splicing pattern, an alternative gene sequence (SEQ ID NO:3) is derived by joining the regions shown as positions 1-215 and 2516-6458 in SEQ ID NO:1. Alternative splicing patterns are known to regulate the synthesis of a variety of peptides and proteins. It may result in proteins with an entirely different function or in dysfunctional or inhibitory splice products (for a review, see McKeown (1992) Annu. Rev. Cell. Biol. 8: 133-155).

The amino acids corresponding to positions 1 to 94 in the published FOXC2 transcription factor (SEQ ID NO:10) are missing in protein encoded by the spliced variant generated from the alternative promoter (SEQ ID NO:4). Consequently, the entire region N-terminal of the DNA binding domain and a portion of the DNA-binding domain (corresponding to positions 72-94 in SEQ ID NO:2) are not present in the splice variant. It is postulated that this truncation leads to a protein which has a deficient “forkhead” DNA-binding region, and thus has a potential inhibitory function on the biological activities of the FOXC2 protein. This truncated FOXC2 protein may have a role in regulation of FOXC2, and an involvment in adipocyte differentiation and adipogenesis.

### Example 4

### Cloning and Sequencing of the FOXC2 Promoter

The DNA region corresponding to nucleotide 176 to nucleotide 2233 (SEQ ID NO. 1 version 2) has been cloned using nested PCR on human genomic DNA. The PCR was performed according the Herculase™ protocol (Stratagene catalog #600260; http://www.stratagene.com/pcr/herculase.htm) and with the inclusion of 8-10% DMSO.

In the initial reaction, the 5′-primer KRKX131 (CCATTGCCTTCTAGTCGC CTCC; SEQ ID NO:14) was used together with the 3′-primer KRKX133 (CGTTGGGG TCGGACACGGAGTA; SEQ ID NO:15) using 250 ng Clontech Genomic DNA #6550-1 as template. The nested reaction was performed on 1/100 of the initial PCR reaction using the 5′-primer KRKX132 (GGTACCTACGCAGCCGATGAACAGCCA; SEQ ID NO:16) and the 3′-primer KRKX134 (GCTAGCGCTGCTTCCGAGACGGCTCG; SEQ ID NO:17). After the second PCR, the product was analyzed by electrophoresis in a 1.2% agarose gel, and a PCR product of the expected size was obtained and extracted for ligation into a TOPO PCR2.1 vector (Invitrogen, Carlsbad, Calif.) by standard cloning procedures and thereafter sequenced. The PCR reaction and cloning procedure was repeated in two parallel separate experiments, and sequence data from the two separate reactions were compared with the bioinformatically assembled sequence.

A DNA region containing the promoter (FIG. 4) corresponding to nt1179 to 2233 (SEQ ID NO:1, version 2) was has been cloned using nested PCR in the same manner as described above. In the initial reaction, the 5′-primer KRKX136 (GGTACCCCCCGAGCC TGGAAACTCCCT; SEQ ID NO:18) was used together with the 3′-primer KRKX134 (GCTAGCGCTGCTTCCGAGACGGCTCG; SEQ ID NO:17) using 250 ng genomic DNA as a template. The PCR reaction and cloning procedure was repeated in four parallel separate experiments, and sequence data from the four separate reactions were compared with the bioinformatically assembled sequence.

### Example 5

### Tissue Expression Profiling of the Alternative Transcript

A reverse transcriptase PCR (RT-PCR) approach was used in order to detect expression of the alternative transcript in human adipose tissue and human primary adipocytes. RNA samples from human adipose tissue (Invitrogen, D6005-01) and primary adipocytes (Zen-Bio, SA75, RNA prepared according to the Trizol protocol)) were analyzed. RT-PCR was performed according to SMART RACE protocol (Clontech). First strand cDNA synthesis was made using a oligo dT primer provided in the SMART RACE kit. For PCR amplification of the alternative transcript, nested 5′ primer specific for the alternative transcript was used (initial PCR step ROLX56 5′ATG AAC AGC CAG GAA GGG TGC AAG G3′ (SEQ ID NO:19) and nested primer ROLX58 5′ACA GCC AGG AAG GGT GCA AGG AAA C3′ (SEQ ID NO:20)) while the nested 3′ primers anneals to sequence common for both the alternative and the normal transcript (initial PCR step ROLX57 5′GAA GCT GCC GTT CTC GAA CAT GTT G 3′ (SEQ ID NO:21) and nested primer ROLX59 5′GTA GGA GTC CGG GTC CAG GGT CCA G 3′ (SEQ ID NO:22)). PCR was performed using the SMART RACE protocol. The primers anneal to sequence on either side of the suggested splice site. Thus a PCR product of the expected size of 223 bp was obtained when amplifying cDNA derived from the alternative transcript, while amplification of contaminating genomic DNA containing the intron sequence yielded a PCR product of much larger size. Using this approach, expression of the alternative transcript was detected in human adipose tissue and primary adipocytes. Expression of the alternative gene product (SEQ ID NO:4) in adipocytes and adipose tissue may be indicative of a regulatory function in this cell type.

### Example 6

### Mapping of the 5′-UTR of the Alternative Exon using cDNA Walking

A cDNA walking method was used in order to map the 5′-UTR of the alternative exon. Human adipose total RNA was obtained from Invitrogen (D6005-01). First strand 5′ RACE cDNA was synthesized according to standard procedure as described in the Clontech manual. The cDNA was amplified according to the manual but using gene specific primers. The 3′-PCR primers used in all reactions anneals to a sequence at the 3′-end of the splice site. Amplification of contaminating genomic DNA yields a PCR product of a larger size, as this would contain the intron sequence. The 5′-PCR primers anneals to sequence upstream of the putative initiation codon of the alternative exon, with approximate 100 bp intervals. PCR products were subsequently cloned using TA cloning in a TOPO vector (Invitrogen) according to manual, and sequenced using standard procedure.

In the PCR reaction yielding the longest PCR product nested 5′-primers were used (initial PCR step 5′-GCGTTCGGCTCACTGACTTACAAGGT-3′ (SEQ ID NO:23) and nested primer 5′-GGAAGTGTCTCTCTCACCTTTTCTGTCTTGA-3′ (SEQ ID NO:24)) together with nested 3′-primers (initial PCR step 5′-GAAGCTGCCGTTCTCGAACA TGTTG-3′ (SEQ ID NO:21) and nested primer 5′-GTAGGAGTCCGGGTCCAGGG TCCAG-3′ (SEQ ID NO:22)). This results in a PCR product of 878 bp (SEQ ID NO:13) containing the predicted sequence. PCR using primers annealing to sequence 5′ of GCGTTCGGCTCACTGACTTACAAGGT (SEQ ID NO:23) does not yield a detectable PCR product. These results suggest that the transcription initiation site for the alternative transcript is located at least 878 bp upstream of the suggested translational start. Position 692 in SEQ ID NO:13 corresponds to position 1 in SEQ ID NO:3.

### Example 7

### Functional Analysis

The identified regulatory regions are analyzed to determine their impact on the transcription of the FOXC2 gene or a reporter gene substituted for FOXC2. A PCR reaction is performed to isolate the promoter region adjacent to the published exon sequence, possibly including the sequences extending to the beginning of the ATG encoding the first methionine. This PCR product is cloned into a reporter plasmid adjacent to a reporter gene (e.g. luciferase). The upstream regulatory region, i.e. regions containing both upstream and promoter proximal sequences, or these sequences bearing artificially induced differences, are cloned in a similar manner. These constructs are transfected into a cell culture model system and the level/activity of the protein encoded by the reporter gene is determined. This would provide information on the function of the identified regions, and used to assess the impact of the different regions on transcriptional regulation. Similarly, the upstream regulatory region, a region containing both upstream and promoter proximal sequences, or these sequences bearing artificially induced differences can be cloned and used to assess the impact of these regions on the transcription of the reporter gene.

### Example 8

### Reporter Gene Assay to Identify Modulating Compounds

Reporter gene assays are well known as tools to signal transcriptional activity in cells. (For a review of chemiluminescent and bioluminescent reporter gene assays, see Bronstein et al. (1994) Analytical Biochemistry 219, 169-181.) For instance, the photoprotein luciferase provides a useful tool for assaying for modulators of promoter activity. Cells are transiently transfected with a reporter construct which includes a gene for the luciferase protein downstream from the FOXC2 promoter and enhancer region, or fragments thereof regulating the FOXC2 activity. Luciferase activity may be quantitatively measured using e.g. luciferase assay reagents that are commercially available from Promega (Madison, Wis.). Differences in luminescence in the presence versus the absence of a candidate modulator compound are indicative of modulatory activity.

TABLE I
Summary of FOXC2 sequences

| SEQ ID | GenBank | |
|---|---|---|
| NO: | accession no. | Description |
| 1 | | Human FOXC2 extended genomic DNA sequence |
| 2 | | Human FOXC2 polypeptide sequence |
| | | (Identical with SEQ ID NO:10) |
| 3 | | Human FOXC2 DNA sequence |
| | | Alternative splicing |
| 4 | | Human polypeptide sequence |
| | | Alternative open reading frame |
| 5 | Y08222 | Mouse MHF-1 (FoxC2) genomic DNA sequence |
| | | (CDS 2070-3554) |
| 6 | X74040 | Mouse MHF-1 (FoxC2) cDNA sequence |
| 7 | | Mouse MHF-1 (FoxC2) polypeptide sequence |
| 8 | Y08223 | Human FKHL14 (FOXC2) genomic DNA |
| | | sequence (CDS 1197-2702) |
| 9 | NM_005251 | Human FKHL14 (FOXC2) cDNA sequence |
| 10 | | Human FKHL14 (FOXC2) polypeptide sequence |
| 11 | AW 271272 | Human EST |
| 12 | AW 793237 | Human EST |
| 13 | | 5′-UTR of the alternative splice variant |

TABLE II
Summary of features in human FOXC2 sequences
shown as SEQ ID NOs: 1 and 3

| | |
|---|---|
| Feature | Positions |

SEQ ID NO:1

| | |
|---|---|
| First exon according to the alternative transcript | 1-215 |
| Untranslated region | 1-186 |
| Region coding for 5′-part of alternative protein | 187-215 |
| Alternative first exon splice site | 215-216 |
| Predicted enhancer region | 216-475 |
| E-box-like region | 223-231 |
| Forkhead-like region | 359-375 |
| Forkhead-like region | 378-402 |
| Ets-like region | 403-423 |
| Predicted promoter region | 1250-1749 |
| Forkhead-like region | 1692-1703 |
| First exon according to the published form of the transcript | 1746-4629 |
| Untranslated region | 1746-2234 |
| Polypeptide coding region | 2235-3740 |
| Region coding for DNA-binding domain | 2448-2735 |
| Second exon according to the alternative transcript | 2516-4629 |
| Portion of polypeptide used in alternative transcript | 2516-3740 |
| Untranslated region | 3741-4629 |

SEQ ID NO:3

| | |
|---|---|
| Polypeptide coding region (5′ of splice site) | 187-215 |
| Polypeptide coding region (3′ of splice site) | 216-1437 |
| Region coding for truncated portion of protein | 216-435 |

What is claimed is:

1. An isolated human FOXC2 promoter region comprising a nucleotide sequence selected from the group consisting of:
(a) nucleotides 1692 to 1703 of SEQ ID NO:1, or a fragment thereof exhibiting FOXC2 promoter activity;
(b) a sequence complementary to (a); and
(c) the sequence of a nucleic acid capable of hybridizing, under stringent hybridization conditions, to a nucleotide sequence as defined in (a) or (b).

2. The human FOXC2 promoter region of claim 1, comprising a nucleotide sequence selected from the group consisting of:
(a) nucleotides 1250 to 1749 of SEQ ID NO:1, or a fragment thereof exhibiting FOXC2 promoter activity;
(b) a sequence complementary to (a); and
(c) the sequence of a nucleic acid capable of hybridizing, under stringent hybridization conditions, to a nucleotide sequence as defined in (a) or (b).

3. The human FOXC2 promoter region of claim 2, comprising a nucleotide sequence selected from the group consisting of:
(a) nucleotides 1250 to 2235 of SEQ ID NO:1, or a fragment thereof exhibiting FOXC2 promoter activity;
(b) a sequence complementary to (a); and
(c) the sequence of a nucleic acid capable of hybridizing, under stringent hybridization conditions, to a nucleotide sequence as defined in (a) or (b).

4. A recombinant construct comprising the human FOXC2 promoter region of claim 1.

5. The recombinant construct of claim 4, wherein the human FOXC2 promoter region is operably linked to a nucleic acid molecule comprising a nucleotide sequence that encodes a detectable product.

6. The recombinant construct of claim 5, wherein the detectable product is a FOXC2 polypeptide.

7. The recombinant construct of claim 4, further comprising a reporter gene.

8. A vector comprising the recombinant construct of claim 4.

9. A host cell stably transformed with the recombinant construct of claim 4.

10. A method for identification of an agent regulating FOXC2 promoter activity, the method comprising:
(i) contacting a candidate agent with the human FOXC2 promoter region of claim 1; and
(ii) determining whether the candidate agent modulates FOXC2 promoter activity.

11. A method for identification of an agent that regulates FOXC2 promoter activity, the method comprising assaying reporter gene expression in the presence of a candidate agent in a cell stably transformed with the recombinant construct of claim 7, wherein an effect on the level of expression of the reporter gene in the presence of the candidate agent as compared to the level of expression of the reporter gene in the absence of the candidate agent indicates that the agent regulates FOXC2 promoter activity.

12. An isolated human FOXC2 enhancer region comprising a nucleotide sequence selected from the group consisting of:
(a) nucleotides 223 to 231 of SEQ ID NO:1, or a fragment thereof exhibiting FOXC2 enhancer activity;
(b) a sequence complementary to (a); and
(c) the sequence of a nucleic acid capable of hybridizing, under stringent hybridization conditions, to a nucleotide sequence as defined in (a) or (b).

13. An isolated human FOXC2 enhancer region comprising a nucleotide sequence selected from the group consisting of:
(a) nucleotides 359 to 375 of SEQ ID NO:1, or a fragment thereof exhibiting FOXC2 enhancer activity;
(b) a sequence complementary to (a); and
(c) the sequence of a nucleic acid capable of hybridizing, under stringent hybridization conditions, to a nucleotide sequence as defined in (a) or (b).

14. An isolated human FOXC2 enhancer region comprising a nucleotide sequence selected from the group consisting of:
(a) nucleotides 378 to 402 of SEQ ID NO:1, or a fragment thereof exhibiting FOXC2 enhancer activity;
(b) a sequence complementary to (a); and
(c) the sequence of a nucleic acid capable of hybridizing, under stringent hybridization conditions, to a nucleotide sequence as defined in (a) or (b).

15. An isolated human FOXC2 enhancer region comprising a nucleotide sequence selected from the group consisting of:
(a) nucleotides 403 to 423 in SEQ ID NO:1, or a fragment thereof exhibiting FOXC2 enhancer activity;
(b) a sequence complementary to (a); and
(c) the sequence of a nucleic acid capable of hybridizing, under stringent hybridization conditions, to a nucleotide sequence as defined in (a) or (b).

16. The human FOXC2 enhancer region of claim 12, comprising a nucleotide sequence selected from the group consisting of:
(a) nucleotides 216 to 475 of SEQ ID NO:1, or a fragment thereof exhibiting FOXC2 enhancer activity;
(b) a sequence complementary to (a); and
(c) the sequence of a nucleic acid capable of hybridizing, under stringent hybridization conditions, to a nucleotide sequence as defined in (a) or (b).

17. A recombinant construct comprising a human FOXC2 enhancer region of claim 12.

18. A vector comprising the recombinant construct of claim 17.

19. A host cell stably transformed with the recombinant construct of claim 18.

20. A method for identification of an agent that regulates FOXC2 enhancer activity, the method comprising:
contacting a candidate agent with the human FOXC2 enhancer region of claim 12; and
determining whether the candidate agent modulates FOXC2 enhancer activity.

21. A method for identification of an agent that regulates FOXC2 enhancer activity, the method comprising assaying reporter gene expression in the presence of a candidate agent in a cell stably transformed with the recombinant construct of claim 17, wherein an effect on the level of expression of the reporter gene in the presence of the candidate agent as compared to the level of expression of the reporter gene in the absence of the candidate agent indicates that the agent regulates FOXC2 enhancer activity.

22. A method for identification of an agent that regulates a mammalian FOXC2 promoter activity, the method comprising:
contacting an isolated nucleic acid sequence with a candidate agent, wherein the nucleic acid sequence comprises a murine FoxC2 promoter nucleotide sequence shown as positions 1250 to 2235 in SEQ ID NO:5; and
determining whether the candidate agent modulates expression of a nucleotide sequence operably linked to the murine FoxC2 promoter nucleotide sequence, such modulation indicating that the agent regulates mammalian FOXC2 promoter activity.

23. A method for identification of an agent that regulates a mammalian FOXC2 enhancer activity, the method comprising:
contacting an isolated nucleic acid sequence with a candidate agent, wherein the nucleic acid sequence comprises a murine FoxC2 enhancer nucleotide sequence shown as positions 216 to 475 in SEQ ID NO:5; and
determining whether the candidate agent modulates expression of a nucleotide sequence operably linked to the murine FoxC2 enhancer nucleotide sequence, such modulation indicating that the agent regulates mammalian FOXC2 enhancer activity.

24. A method for identification of an agent that regulates a mammalian FOXC2 enhancer activity, the method comprising:
contacting an isolated nucleic acid sequence with a candidate agent, wherein the nucleic acid sequence comprises a murine FoxC2 enhancer nucleotide sequence shown as positions 216 to 2235 in SEQ ID NO:5; and
determining whether the candidate agent modulates expression of a nucleotide sequence operably linked to the murine FoxC2 enhancer nucleotide sequence, such modulation indicating that the agent regulates mammalian FOXC2 enhancer activity.

25. An isolated nucleic acid molecule comprising a nucleotide sequence selected from the group consisting of:
(a) SEQ ID NO:3 or a complement thereof;
(b) the sequence of a nucleic acid capable of hybridizing, under stringent hybridization conditions, to a nucleotide sequence complementary to the polypeptide coding region of a nucleic acid molecule as defined in (a) and which codes for a variant form of the FOXC2 transcription factor;
(c) the sequence of a nucleic acid which is degenerate as a result of the genetic code to a nucleotide sequence as defined in (a) or (b) and which codes for a variant form of the FOXC2 transcription factor; and
(d) a nucleic acid that encodes the polypeptide of SEQ ID NO:4.

26. An isolated polypeptide comprising a polypeptide sequence encoded by the nucleic acid molecule of claim 25.

27. The isolated polypeptide of claim 26, wherein the polypeptide comprises the amino acid sequence of SEQ ID NO:4.

28. A vector comprising the nucleic acid molecule of claim 25.

29. A replicable expression vector, that carries and is capable of mediating expression of the nucleotide sequence of claim 25.

30. A cultured host cell comprising the vector of claim 28.

31. A process for the production of a variant form of the FOXC2 transcription factor polypeptide, the process comprising culturing the host cell of claim 30 under conditions whereby the polypeptide is produced, and recovering the polypeptide.

32. A method for identifying an agent that regulates expression of the nucleic acid molecule of claim 25, said method comprising:
contacting a candidate agent with the nucleic acid molecule; and
determining whether said candidate agent modulates expression of the nucleic acid molecule.

33. An antisense oligonucleotide having a sequence capable of specifically hybridizing to RNA transcribed from the nucleic acid molecule of claim 25, so as to prevent translation of the RNA.

34. A method for the identification of a polypeptide that modulates the activity of a FOXC2 nucleotide sequence, comprising:
(a) transfecting a cell line with a human FOXC2 nucleotide sequence operably linked to a reporter gene;
(b) transfecting the cell line with a plurality of human cDNA sequences;
(c) identifying and isolating transfected cells having an altered level of expression of the reporter gene, as compared to cells that have not been transfected with the human cDNA sequences;
(d) recovering cDNA from the isolated cells isolated in step (c); and
(e) identifying the polypeptide encoded by the cDNA recovered in step (d).

35. A nucleic acid comprising a nucleotide sequence selected from the group consisting of nucleotides 1692 to 1703 of SEQ ID NO:1, nucleotides 223 to 231 of SEQ ID NO:1, nucleotides 359 to 375 of SEQ ID NO:1, nucleotides 378 to 402 of SEQ ID NO:1, and nucleotides 403 to 423 in SEQ ID NO:1, operably linked to a heterologous coding sequence.
